(19) United States
(12) Patent Application Publication
(10) Pub. No.: US 2025/0281210 A1
(43) Pub. Date: Sep. 11, 2025

(54) SYSTEMS AND METHODS FOR TREATING RIB FRACTURES AND OSTEOTOMIES USING IMPLANTATION

(71) Applicant: GLOBUS MEDICAL, INC., AUDUBON, PA (US)

(72) Inventors: John LaColla, West Chester, PA (US); Jeff Nichols, Medford, NJ (US); Matthew McClelland, Phoenixville, PA (US)

(21) Appl. No.: 19/092,035

(22) Filed: Mar. 27, 2025

Related U.S. Application Data
(63) Continuation-in-part of application No. 18/345,167, filed on Jun. 30, 2023.
Application No. 18/345,167 is a continuation of application No. 17/112,134, filed on Dec. 4, 2020, now Pat. No. 11,707,307.
(60) Provisional application No. 63/570,911, filed on Mar. 28, 2024.

(51) Int. Cl. A61B 17/80; A61B 17/17; A61B 17/70

(52) U.S. Cl. CPC A61B 17/8076; A61B 17/1728; A61B 17/707

(57) ABSTRACT

Systems and methods include solutions for fixation at the rib head for fractures and osteotomies adjacent to the rib head and transverse process. The disclosed rib plates, anchor systems, other implants, and instrumentation may also be applied to mid-rib fractures. The systems and methods may be used in the treatment of rib deformities, including the correction of rib hump deformity via thoracoplasty, as well as general corrections of chest and rib deformities. Systems and methods herein may be used in chest wall reconstructions due to trauma, cancer, or deformity.

### CROSS-REFERENCE TO RELATED APPLICATIONS

The present application claims the benefit of priority of U.S. Provisional Patent Application No. 63/570,911, filed on Mar. 28, 2024. The present application is a continuation-in-part of U.S. patent application Ser. No. 18/345,167, filed Jun. 3, 2023 (published as U.S. Pat. Pub. No. 2024-0008906), which is a continuation of U.S. patent application Ser. No. 17/112,134, filed on Dec. 4, 2020. All of the aforementioned patent applications are each incorporated herein by reference in their entireties for all purposes.

### FIELD

The present disclosure relates to systems and devices for treating fractures and osteotomies of ribs. More particularly, the disclosure relates to implant assemblies for fixating the ribs to aid in the healing process.

### BACKGROUND

Traditionally, fractures and osteotomies of the ribs were left untreated to heal, or were occasionally aided by casting. Some surgeons may have used sutures to reduce migration of the affected bone segments. More recently, rib fixation systems have become available, and early data and accounts suggest that the reduced motion and increased stability may reduce the pain associated with healing.

Current rib fixation devices are often plate-style, using screws or other anchors to fixate across a fracture site. These methods are sufficient for fractures and osteotomies which occur mid-rib and have adequate bone available on either side of the fracture for fixation, typically two (2) or three (3) screws on either side. In the case of posterior rib fractures adjacent to the rib head, the existing methods are inadequate due to lack of fixation.

What is needed are rib fixation solutions for fixation at the rib head, for fractures and osteotomies adjacent to the rib head and transverse process. What is also needed are fixation solutions that may be applied to mid-rib fractures, making them more versatile and capable of treating a wider range of pathologies than existing methods.

### SUMMARY

According to one embodiment, a system for treating a fracture of a rib including a plate adapted to be attached to the rib, the plate including a plurality of bone fastener holes, each of the plurality of bone fastener holes configured to receive a bone fastener. The system also includes an implant holder having an inferior surface configured to engage an inferior surface of the rib and superior surface, a drill guide including one or more cannulas and a first threaded portion, and an inserter instrument having a central shaft and a distal tip. The distal tip is configured to engage bone, and the inserter instrument has a second threaded portion configured to engage and mate with the first threaded portion.

According to one embodiment, a system for treating a fracture of a rib including a plate including a plurality of bone fastener holes, at least one bone fastener configured to be received in one of the plurality of bone fastener holes, an implant holder having an inferior surface configured to engage an inferior surface of the rib and a superior surface, a drill guide including one or more cannulas and a first threaded portion, and an inserter instrument having a central shaft and a distal tip. The distal tip is configured to engage bone and the inserter instrument has a second threaded portion configured to engage and mate with the first threaded portion.

### BRIEF DESCRIPTION OF THE DRAWINGS

The accompanying drawings, which are incorporated herein and constitute part of this specification, illustrate the presently preferred embodiments of the disclosure, and, together with the general description given above and the detailed description given below, serve to explain the features of the disclosure. In the drawings:

FIG. 1A illustrates an exemplary embodiment of a rib plate consistent with the principles of the present disclosure.

FIG. 1B illustrates an exemplary embodiment of a rib plate consistent with the principles of the present disclosure.

FIG. 1C illustrates an exemplary embodiment of a rib plate consistent with the principles of the present disclosure.

FIG. 2 illustrates an exemplary embodiment of a rib plate consistent with the principles of the present disclosure.

FIG. 3A illustrates an exemplary embodiment of a rib plate consistent with the principles of the present disclosure.

FIG. 3B illustrates an exemplary embodiment of a rib plate consistent with the principles of the present disclosure.

FIGS. 4A-4F illustrate an exemplary embodiment of a rib head anchor consistent with the principles of the present disclosure.

FIGS. 5A and 5B illustrate an exemplary embodiment of a rib head anchor consistent with the principles of the present disclosure.

FIGS. 6A-6B illustrate an exemplary embodiment of splint plate consistent with the principles of the present disclosure.

FIGS. 7A-7C illustrate an exemplary embodiment of a rib head anchor consistent with the principles of the present disclosure.

FIG. 7D illustrates an exemplary embodiment of a rib head anchor with a longitudinal member attached therein consistent with the principles of the present disclosure.

FIGS. 8A and 8B illustrate an exemplary embodiment of a rib head anchor system consistent with the principles of the present disclosure.

FIGS. 9A and 9B illustrate an exemplary embodiment of a rib plate consistent with the principles of the present disclosure.

FIGS. 10A-10C illustrate an exemplary embodiment of a rib plate and a screw guide for fixating the plate to a bone consistent with the principles of the present disclosure.

FIGS. 11A and 11B illustrate an exemplary embodiment of a rib plate consistent with the principles of the present disclosure.

FIGS. 12A and 12B illustrate an exemplary embodiment of a rib plate consistent with the principles of the present disclosure.

FIGS. 13A and 13B illustrate an exemplary embodiment of a rib head anchor consistent with the principles of the present disclosure.

FIGS. 14A-14H illustrate exemplary embodiments of a capture feature for a rib plate consistent with the principles of the present disclosure.

FIGS. 15A-15C illustrate an exemplary embodiment of locking screw for a rib plate consistent with the principles of the present disclosure.

FIGS. 16A and 16B illustrate exemplary embodiments of a rib plate consistent with the principles of the present disclosure.

FIGS. 17A-17C illustrate an exemplary embodiment of a splint plate consistent with the principles of the present disclosure.

FIGS. 18A-18F illustrate an exemplary embodiment of a rib head screw and L-rod consistent with the principles of the present disclosure.

FIG. 19 illustrates an exemplary embodiment of an inserter instrument consistent with the principles of the present disclosure.

FIGS. 20A-20B illustrate an exemplary embodiment of a drill guide consistent with the principles of the present disclosure.

FIG. 21 illustrates an exemplary embodiment of an inserter instrument engages with a drill guide consistent with the principles of the present disclosure.

FIG. 22 illustrates an exemplary embodiment of an implant holder consistent with the principles of the present disclosure.

FIGS. 23-25 illustrate an exemplary embodiment of an inserter instrument, drill guide, and implant holder consistent with the principles of the present disclosure.

FIGS. 26A-26B illustrate an exemplary embodiment of an implant holder consistent with the principles of the present disclosure.

### DETAILED DESCRIPTION

In the drawings, like numerals indicate like elements throughout, with alphabetical or prime identifiers indicating a particular one of the more generally identified element. Certain terminology is used herein for convenience only and is not to be taken as a limitation on the present invention. The following describes preferred embodiments of the present disclosure. However, it should be understood, based on this disclosure, that the invention is not limited by the preferred embodiments described herein.

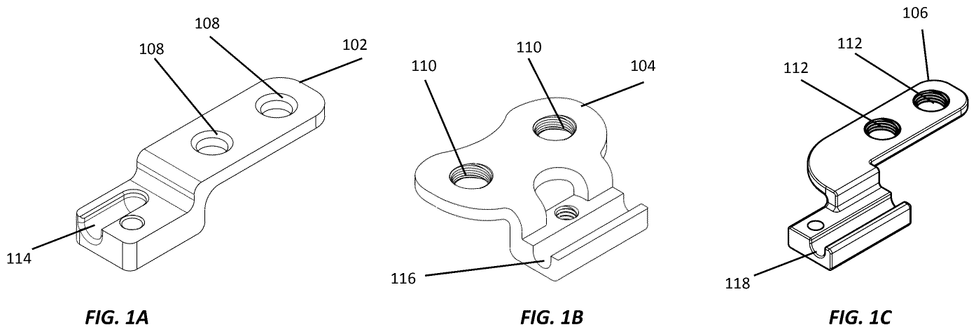

Referring now to FIG. 1A, illustrated is an exemplary embodiment of a rib plate 102 consistent with the principles of the present disclosure. FIGS. 1B and 1C illustrates other exemplary rib plates 104 and 106, respectively. Rib plate 102 may include screw holes 108 to fixate rib plate 102 to a surface of a rib using one or more screws or other anchors, such as sutures. Rib plate 102 may accept a longitudinal member from another implant at portion 114, which may be secured using various methods. The longitudinal member may be inserted in a top-loading manner to a top surface of the rib plate. Rib plate 102 may use a set screw to clamp the longitudinal member in place. Rib plate 110 may have screw holes 110 to accept screws or other anchors. Rib plate 106 may have screw holes 112 for this purpose. The varying geometry of the rib plates may be offset vertically and/or laterally to reduce profile/prominence, as well as to allow uninterrupted growth of the rib.

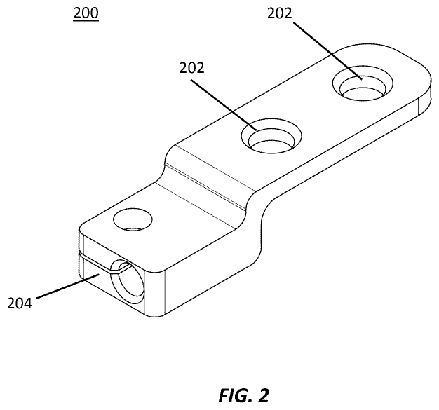

FIG. 2 illustrates a rib plate 200 consistent with the principles of the present disclosure. As with the rib plates of FIGS. 1A-1C, rib plate 200 may have screw holes 202 to fixate rib plate 200 to the surface of the rib, and similarly may be connected to a longitudinal member for stabilization of the fracture or osteotomy. Rib plate 200 has a clamp 204 that may receive the longitudinal member and be secured using a set screw.

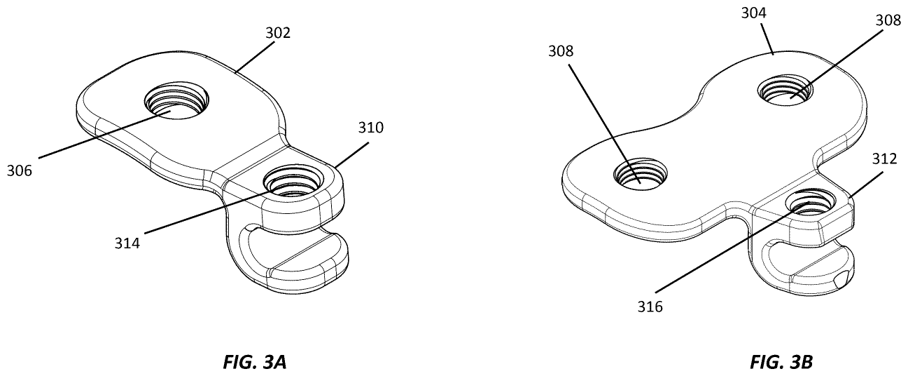

FIGS. 3A and 3B illustrates rib plates 302 and 304 consistent with the principles of the present disclosure. Rib plates 302 and 304 are similar to the other rib plates disclosed herein and fixate on the surface of the rib using screws and anchors received in screw holes 306 and 308, and connect to a longitudinal member via a clamp or locking mechanism 310, 312 offset from the rib, preferably either cephalad or caudal to the rib itself. Clamps 310 and 312 may be offset vertically relative to the plate portion to reduce prominence, and may be angled towards or away from the plate to facilitate access with instruments for tightening or other manipulation.

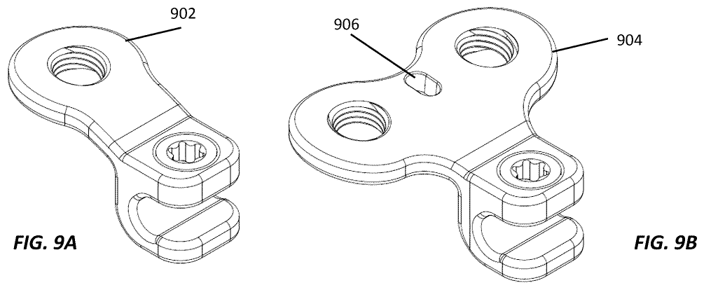

Rib plates 302, 304 connect to a longitudinal member to create stabilization of a fracture or osteotomy. The connections may be made using a set screw received in holes 314 and 316. The rib plate may have one or multiple screws or fixation points for connection to the bone. Rib plate 302 is shown with one fixation point and rib plate 304 is shown with multiple fixation points. An example of usage for the single set screw embodiment of rib plate 302 is to secure a fragment or portion of bone which has been resected or separated from the remainder of the rib, and to hold it in a certain location to function as a graft to reduce the necessary span of bone regrowth. Other related rib plates are illustrated in FIGS. 9A and 9B, showing rib plates 902 and 904.

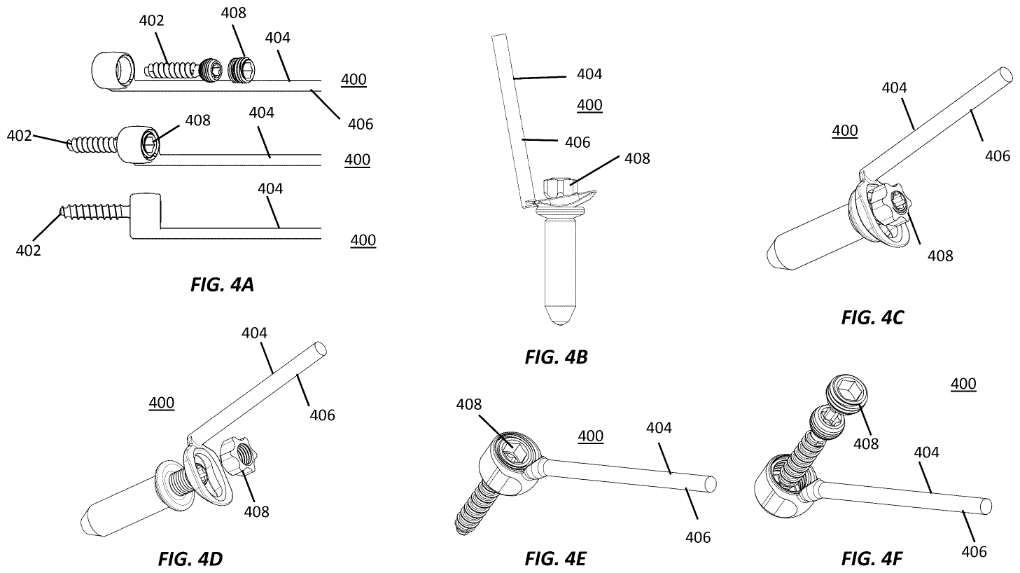

FIGS. 4A-4F show illustrations of a polyaxial rib head anchor 400. Rib head anchor 400 may be implanted within the rib, preferably by driving a screw 402 through the longitudinal/intramedullary axis of the rib. Rib head anchor 400 may be used anywhere in the rib, but likely provides the most utility when implanted in the most proximal portion of the rib, adjacent to the transverse process, after an osteotomy is performed to remove a portion of the rib.

Rib head anchor 400 uses screw 402 assembled with a main body 404 which includes a longitudinal member 406 and a locking mechanism 408. Screw 402 may be preassembled with main body 404 or assembled at the time of the procedure. Locking mechanism 408 is shown as a set screw, but other locking mechanisms may be used.

Rib head anchor 400 is polyaxial allowing variability in angulation and rotation of rib head anchor 400 to accommodate a patient's anatomy and surgical needs. Rib head anchor 400 may be locked by various means, including but not limited to, direct friction, interlocking surfaces, or activation of a clamp or other locking mechanism.

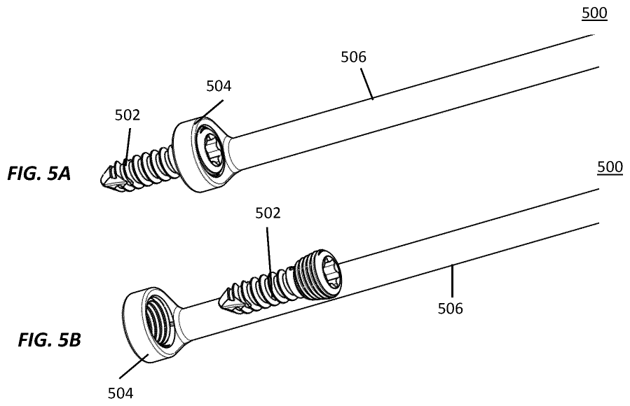

FIGS. 5A and 5B illustrate a fixed rib head anchor 500 having a screw 502, a main body 504 which includes a longitudinal member 506. Rib head anchor 500 is similar to rib head anchor 400 but does not incorporate an adjustable polyaxial mechanism. Rib head anchor 500 is fixed to the rib using a screw 502. Screw 502 and main body 504 incorporate an interlocking mechanism which is activated by seating screw 502 into main body 504. Two examples of a possible interlocking mechanism are monoaxial and polyaxial locking holes.

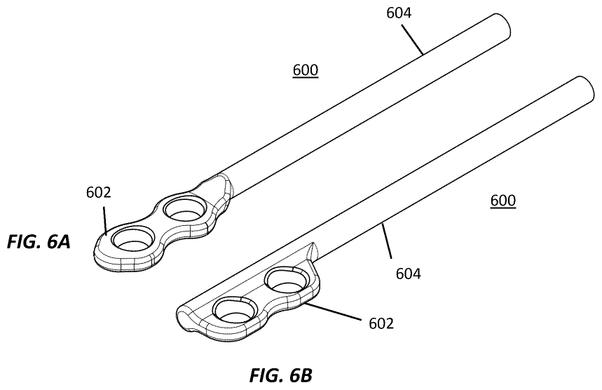

FIG. 6 illustrates a splint plate 600 consistent with the principles of the present disclosure. Splint plate 600 includes a plate portion 602 and a longitudinal member 604. Plate portion 602 may be integrated with longitudinal member 604, for the creation of interconnections between implants, such as implants disclosed herein. Splint plate 600 may include one or more fixation points for connection to the surface of the rib, using bone screws or some other means.

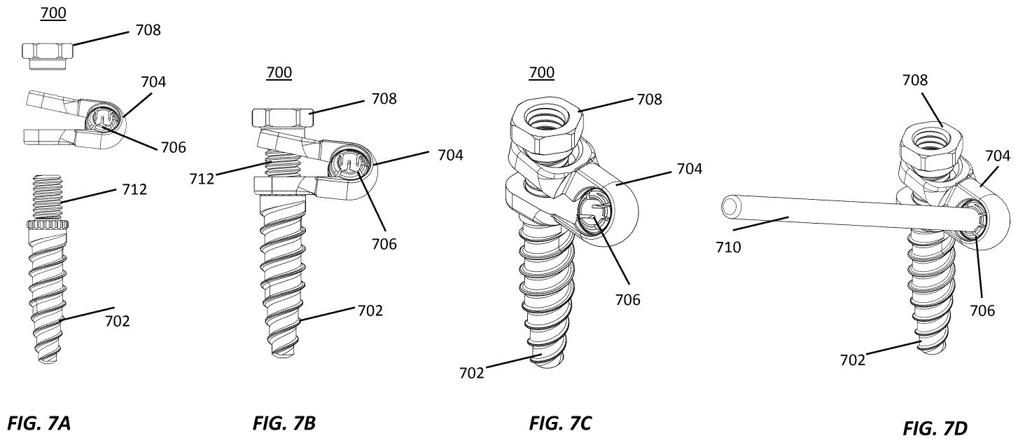

FIGS. 7A-7C illustrate an exemplary polyaxial rib head anchor 700 consistent with the principles of the present disclosure. Rib head anchor 700 may include a bone screw 702, a clamp body 704, a flexible collet 706, and a locking nut 708. Rib head anchor may connect to a longitudinal member 710 as shown in FIG. 7D. Rib head anchor may be used anywhere in the rib including the most proximal portion of the rib, adjacent to the transverse process, after an osteotomy is performed to remove a portion of the rib.

Bone screw 702 may be implanted within the rib, preferably within the longitudinal/intramedullary axis of the rib. Bone screw 702 may include bone threads at a distal tip, a male locking spline in the midsection, and a locking thread at the proximal end, with a driving feature which may mates with a driver for insertion. The bone threads engage with the bone, and the locking spline is left exposed.

Clamp body 704 consists of a split design, in which a flexible collet 706 is assembled. Flexible collet 706 may be spherical and mate into an internal pocket of clamp body 704 of similar size. Collet 706 may be free to rotate in the neutral state of clamp body 704. At the opposite end of clamp body 704 from collet 706 are two through holes which are intended to accept the proximal locking thread geometry of bone screw 702. One of the through holes on clamp body 704 may contain a female locking spline geometry which corresponds to that of bone screw 702. It is noted that while a female locking spline on the clamp body and a corresponding male locking spline on the bone screw are disclosed, this engagement may use other types of mating geometries such as grooves, tapers, or other mating surfaces. When clamp body 704 is placed over bone screw 702 a post 712 of bone screw 702 and the locking splines are engaged, clamp body 704 and bone screw 702 become rotationally linked.

Flexible collet 706 may include a central through hole which may accept longitudinal member 712, such as a round rod, as shown in FIG. 7D. Collet 706 may contain flexures which enable the application of a clamping force to longitudinal member 712. The outer surface of collet 706 may be textured or have locking geometry, which may mate with corresponding textured or locking geometry on clamp body 704.

When longitudinal member 712 is placed into collet 706, and clamp body 704 and collet 706 are placed onto bone screw 702 which has been implanted into the bone, longitudinal member 712 and collet 706 may rotate through a certain range of motion within clamp body 704, enabling assembly of a construct to span an osteotomy or fracture. Locking nut 708 may be introduced to constrain this motion, by engaging with locking threads of bone screw 702, and applying force to the split portion of the clamp body 704. This force causes clamp body 704 to flex and close, and the resulting action provides a clamping force to collet 706, which in turn provides a clamping force to longitudinal member 712.

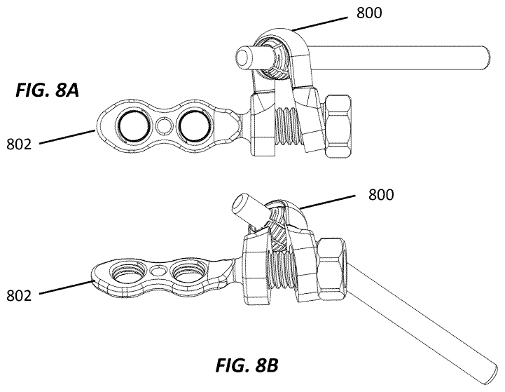

FIGS. 8A and 8B illustrate a rib head anchor system 800 consistent with the principles of the present disclosure. Rib head anchor system 800 is similar to rib head anchor 700 but in place of the bone screw which engages the intramedullary space of the bone, a plate 802 allows for attachment to the outer surface of bony anatomy. The outer surface may be either the outer surface of the rib or the outer surface of the transverse process.

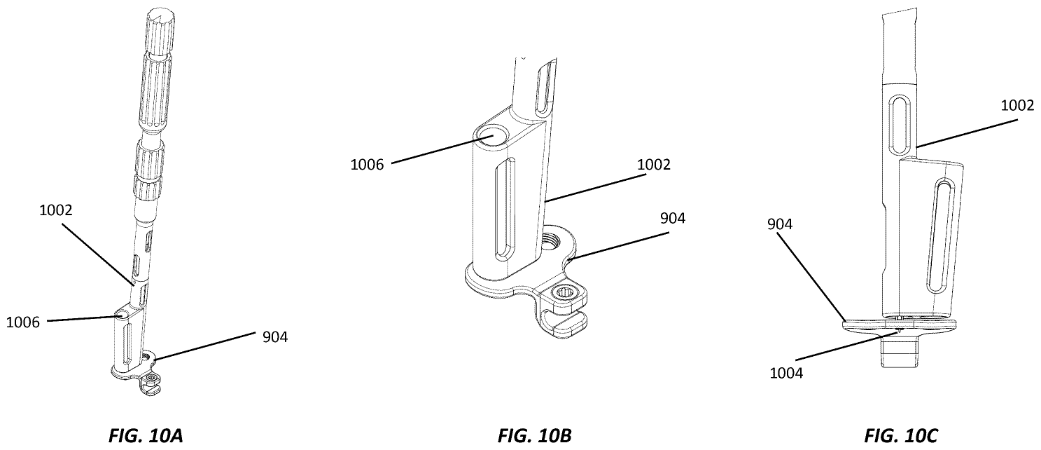

FIGS. 10A-10C illustrates screw guide 1002 consistent with principles of the present disclosure. Screw guide 1002 may be used for plates having a dual screw configuration such as plate 904 shown in FIG. 9B. Plate may have a mating feature 906 to attach to screw guide 1002 to facilitate attachment of plate 904 to bone. Mating feature 904 may be a female slot on the plate that engages with an expanding, split male feature 1004 on screw guide 1002 (shown in FIG. 10C).

Screw guide 1002 may allow the user to position and hold a plate in place while helping to align the appropriate instruments for insertion of the locking bone screw, which is shown in FIGS. 10A-10C in a converging trajectory. A cannula 1006 is provided at the distal end of screw guide 1002 which allows coaxial alignment with screw holes in the plate. The user may use a drill and/or awl to prepare the pathway for the locking bone screw via cannula 1006, and then pass the bone screw which is attached to a driver. After placement of one screw, cannula 1006 may be rotated about the central axis to align with the opposite locking bone screw trajectory for insertion.

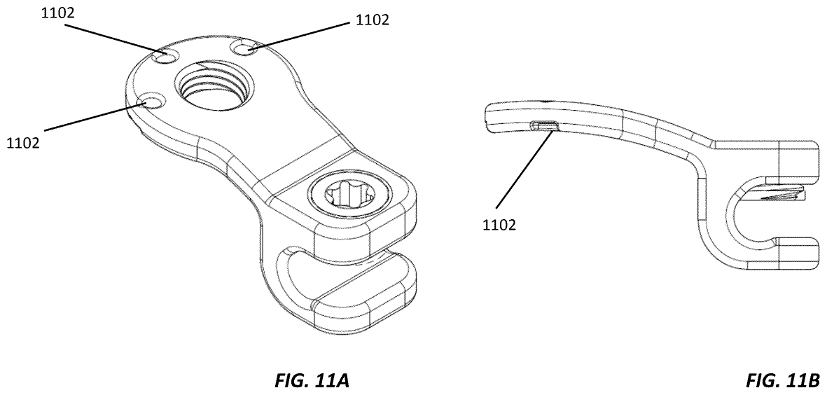

Referring to FIGS. 11A and 11B, the plates described herein may include suture holes 1102 to facilitate attachment of soft tissue to the plate or provide additional support to secure the implant to bony anatomy.

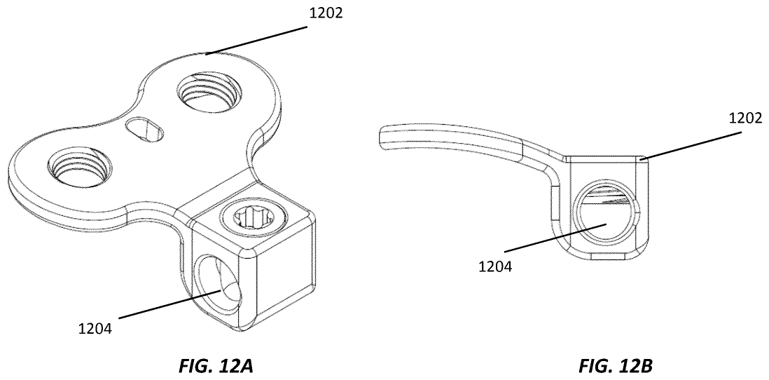

Referring to FIGS. 12A and 12B, illustrated is a plate 1202 in a closed configuration. Any of the plates described herein may be configured in a closed configuration, where a longitudinal member is passed through a clamp portion 1204 of the plate. This may provide additional stability by reducing the potential for the longitudinal member to escape the clamp during manipulation and correction of an osteotomy or fracture.

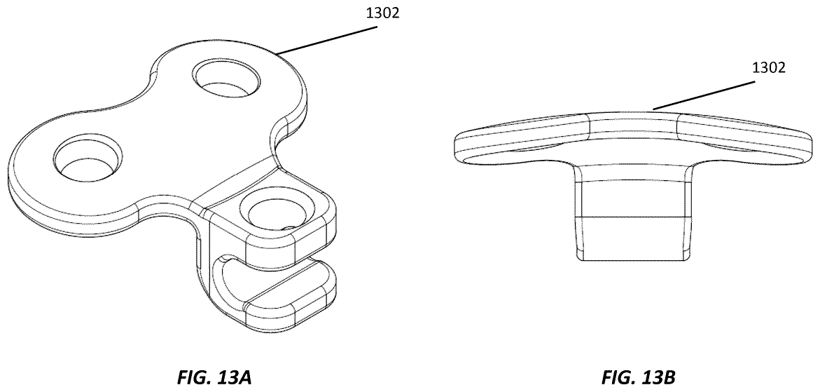

Illustrated in FIGS. 13A and 13B, a plate 1302 may be curved in multiple planes to better accommodate complex geometry of the ribs. Plate 1302 is configured such that a portion of the plate is contoured in both the cephalad-caudal and proximal-distal planes of the rib.

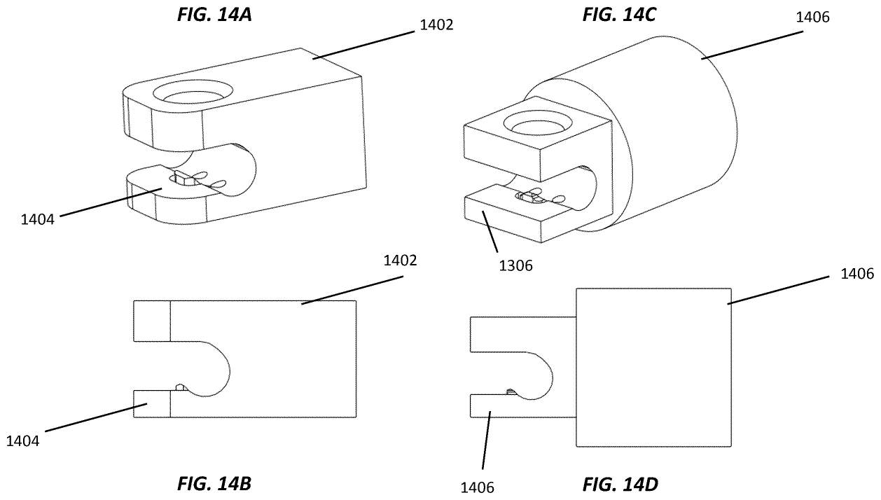

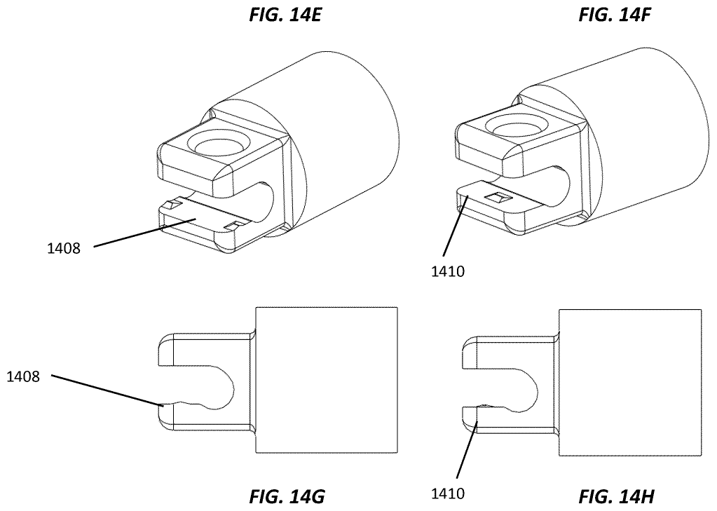

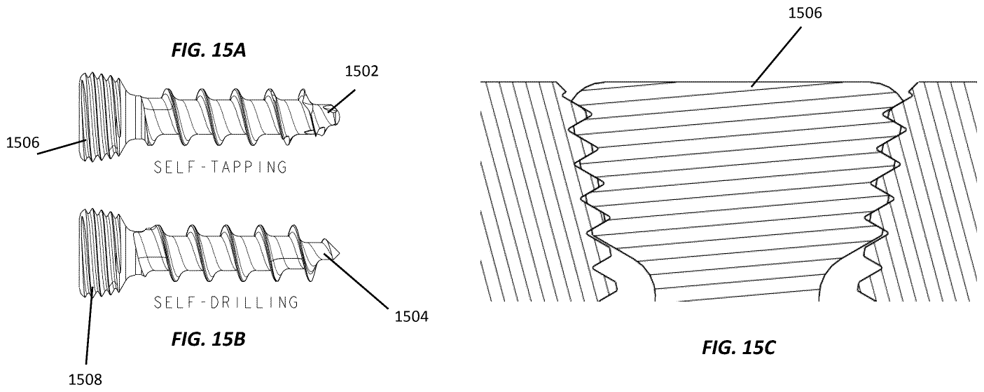

FIGS. 14A-H illustrate provisional capture features 1402 and 1404. FIG. 13B shows a side view of FIG. 13A and FIG. 13D shows a side view of FIG. 13C. This may apply to any of the clamps for plates described herein. The plates may be augmented with the provisional capture features 1404, 1406, which provides tactile and/or audible feedback to the user to indicate that the longitudinal member has been provisionally captured in the clamp. The performance of the provisional capture features 1404, 1406 is accomplished by allowing a portion of the device to flex and allow passage of the longitudinal member.

FIGS. 14A-14D illustrate provisional capture features 1404, 1406. Provisional capture features 1408, 1410 is accomplished by interfering geometry located within the clamp mouth. The magnitude of the interference will be appropriately small such that the longitudinal member may be seated into the clamp by applying force by hand or with an instrument. The interference features may be located on opposing surfaces within the clamp mouth, or may be one-sided, opposing only the normal clamp geometry.

FIGS. 15A-15C illustrate monoaxial locking bone screws for rib plates consistent with the principles of the present disclosure. Screw 1502 may be a self-tapping screw and screw 1504 may be a self-drilling screw. These bone screws may be used with any of the plates described herein to secure the plate to the bony anatomy of the rib surface.

Bone screw 1502 includes a self-tapping configuration having three (3) swept cutting flutes at the distal end. Bone screw 1504 includes a self-drilling configuration having a sharp tip. Both configurations have bone threads at the distal end, with a tapered monoaxial locking head at the proximal end as well as a female drive feature (for example, a hexalobe).

The monoaxial locking geometry may be of a dual-lead tapered thread form, with the male/external thread geometry on the screw head, and the female/internal thread geometry on the corresponding hole on the plate device. Locking is accomplished by driving the screw through the corresponding hole on the plate, causing engagement of the tapered threads and eventual interference of the thread geometry.

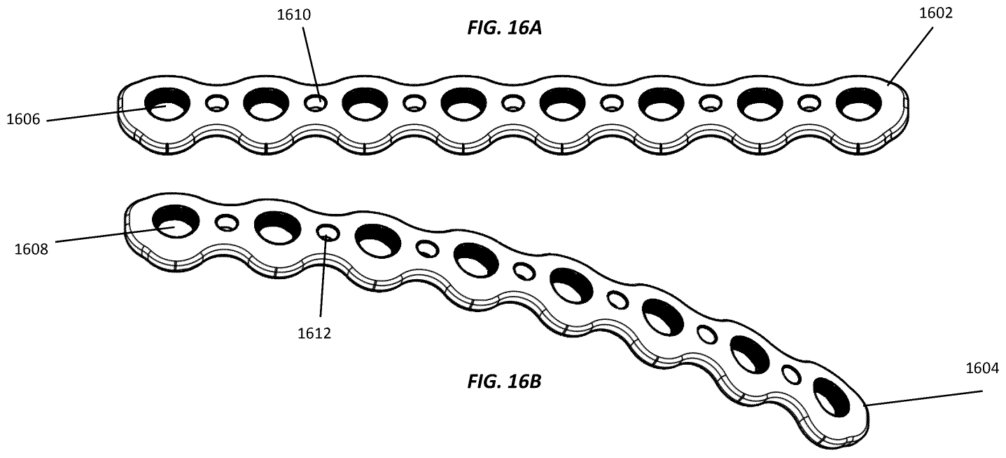

Illustrated in FIGS. 16A and 16B are rib plates 1602 and 1604 consistent with the principles of the present disclosure. Plates 1602 and 1604 may be disposed on the bony surface of the rib and span a fracture or osteotomy. The plate consists of several locking hole features 1606, 1608 along the length of the plate for fixation onto the rib. Holes 1604, 1606 may have the same monoaxial tapered locking geometry as described above. Plates 1602, 1604 may have a scalloped design to facilitate bending in multiple planes. The plates may also include k-wire holes and/or suture holes 1610, 1612. The bottom surface of the plate may be contoured in the cephalad-caudal plane to match the anatomy of the rib, or it may be flat. The plate may be configured in a straight, unbent configuration (FIG. 16A) or a precontoured configuration (FIG. 16B).

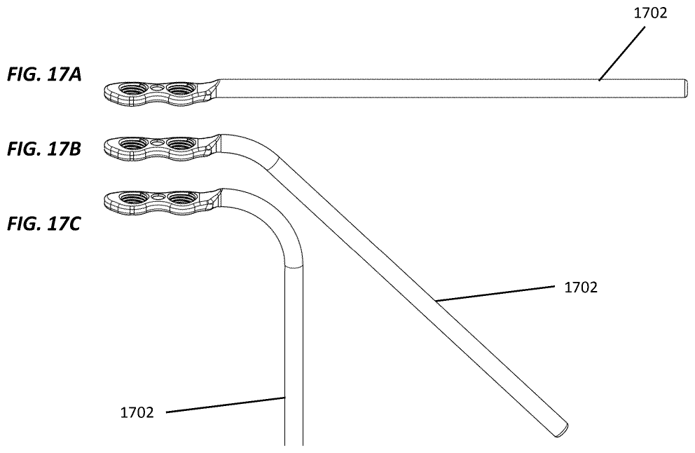

Referring now to FIGS. 17A-17C, illustrated is a splint plate 1702 consistent with the principles of the present disclosure. Here, splint plate 1702 is similar to splint plate 600 of FIGS. 6A and 6B. Splint plate 1702 may include a longitudinal member that is pre-contoured to reduce the amount of bending required by the user during surgery.

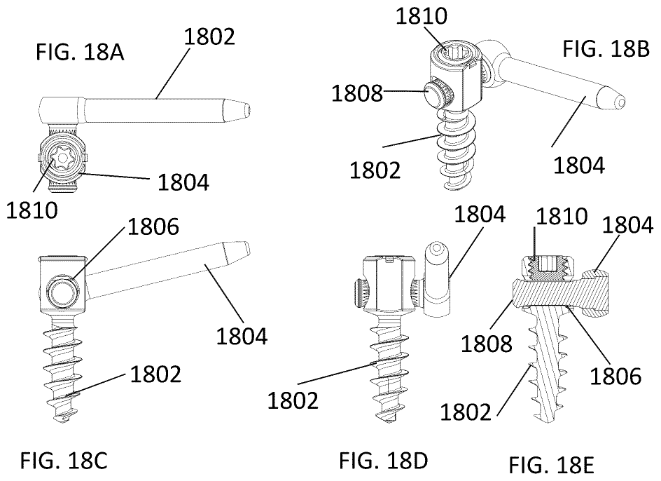

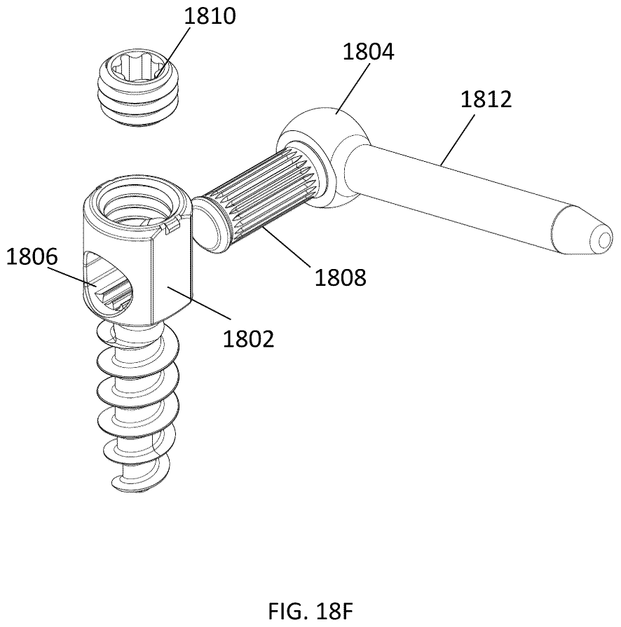

Referring now to FIGS. 18A-18F, illustrated is an embodiment of a rib head screw 1802 and an L-rod 1804 consistent with the principles of the present disclosure. Rib head screw 1802 and L-rod 1804 may have interlocking geometries to increase mating performance between mating parts. These features may be interlocking teeth or other geometry, surface treatments (grit blast, etc), or other design features intended to increase holding force and/or friction between mating parts to improve locking performance between assembled components. In one embodiment shown in FIGS. 18A-18F, male spline/teeth geometry has been added to a through-bore 1806 of screw 1802, and corresponding female spline/teeth geometry has been added a protrusion portion 1808 to L-rod 1804. In the assembled configuration, the spline geometry interlocks and provides additional resistance to movement when a set screw locking mechanism 1810 is tightened, improving performance of screw 1802 and L-rod 1804 under biomechanical loads. It will be understood that L-rod 1804 may have a multi-piece assembly with protrusion 1808 attaching to elongated section 1812 rather than a single bent piece. The components may be joined by any standard manufacturing methods.

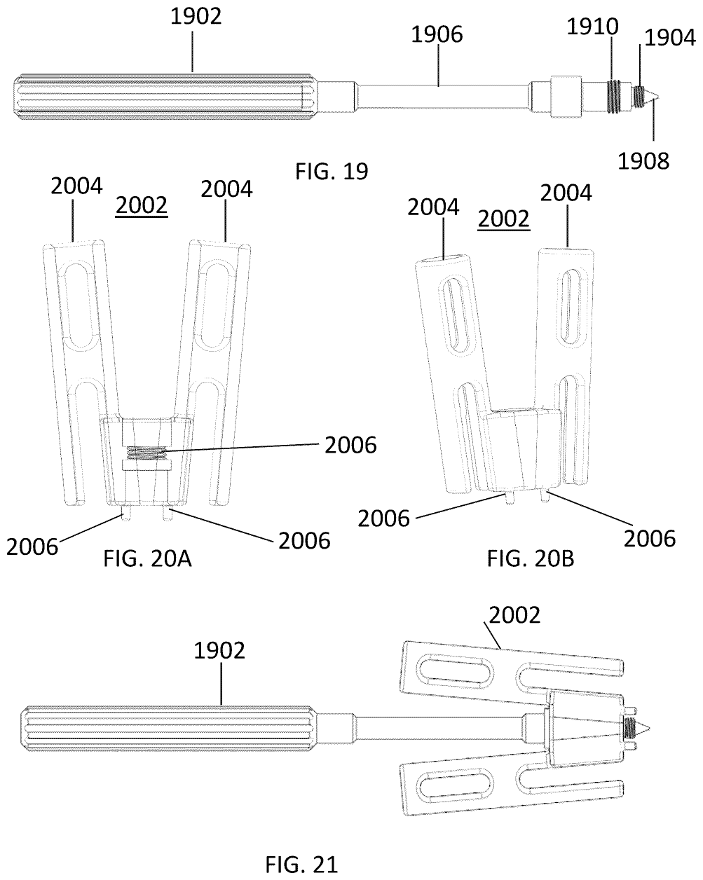

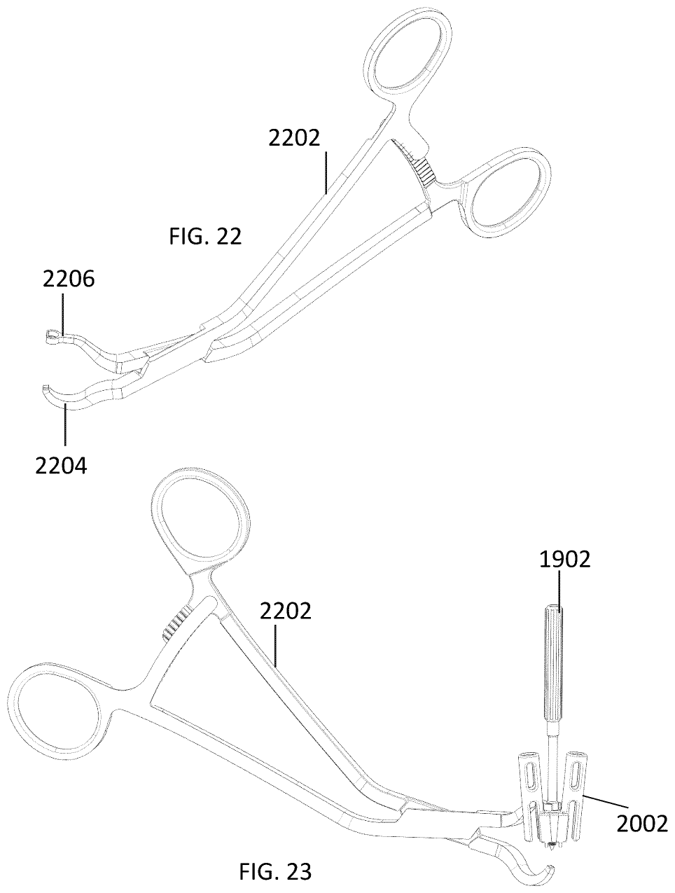

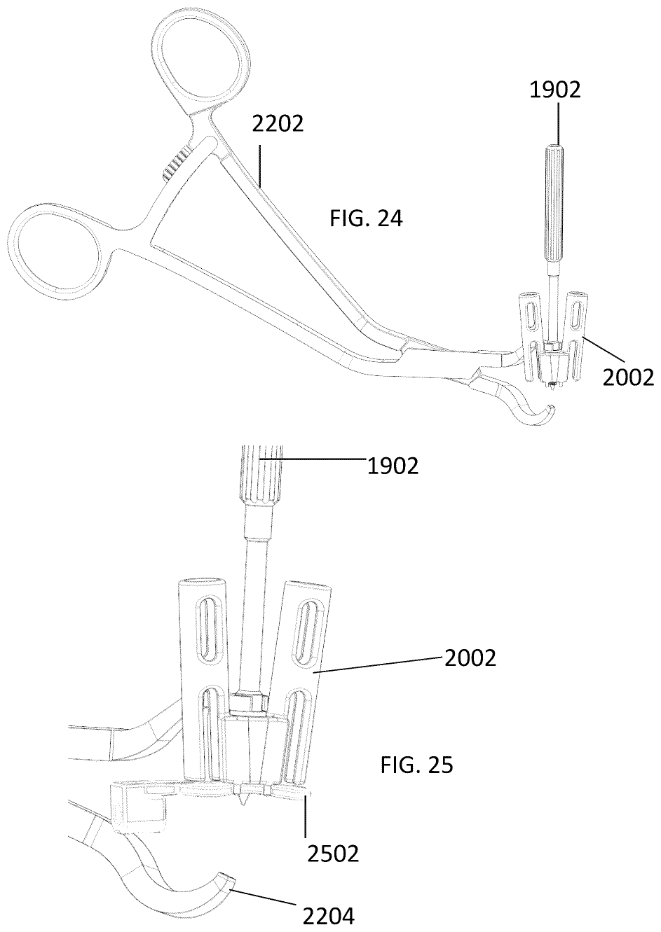

Referring now to FIGS. 19-23, illustrated are exemplary embodiments of an inserter instrument, an implant holder, a drill guide, and plate consistent with principles of the present disclosure. These components may facilitate approximation of the Modular and Lateral plate implants along with screw preparation and insertion.

In the exemplary embodiments shown a threaded hole may be located between the monoaxial screw locking holes of a modular or lateral plate, equidistant from each. The threaded hole is connected to a corresponding threaded feature 1904 on an inserter instrument 1902. Inserter instrument 1902 has a central shaft 1906 to which a drill-screw guide 2002 may be attached for screw preparation (such as drilling) and/or screw insertion. Attachment may be accomplished via connection between a threaded portion 1910 on inserter instrument 1902 and a threaded portion 2006 on drill guide 2002. Drill guide 2002 may also have one or more cannulas 2004 which align with the locking screw holes in a plate 2502 or any plate previously described herein. Drill-screw guide 2002 has geometry which engages with the implant to maintain alignment of cannulas 2004, either by engaging the external contour of the implant, or via features such as a male pin feature 2006 on guide 2002 engaged with a female hole feature on the implant.

Inserter instrument 1902 may have a sharp tip 1908 or other feature located distally to a threaded portion 1904 of central shaft 1906, which engages bony anatomy and helps stabilize the implant on the bone surface during the screw insertion process.

Central shaft 1906 may be used with or without the drill-screw guide 2002 to fix the implant to the bony anatomy. Central shaft 1906 may be used in conjunction with an implant holder 2202. In exemplary embodiments, implant holder 2202 is a forcep-style instrument. The inferior portion of implant holder 2202 (bottom forcep jaw 2204) has a flat surface, sharp point, or other geometry which engages an inferior surface of the rib, opposite a superior surface on which the implant is to be placed. A superior portion 2206 of implant holder 2202 may have features which engage central shaft 1906 and are sized only to allow disengagement along certain portions of central shaft 1906. Implant holder 2202 may be configured to apply a compressive load via central shaft 1906 to the implant, fixturing it to the bony surface.

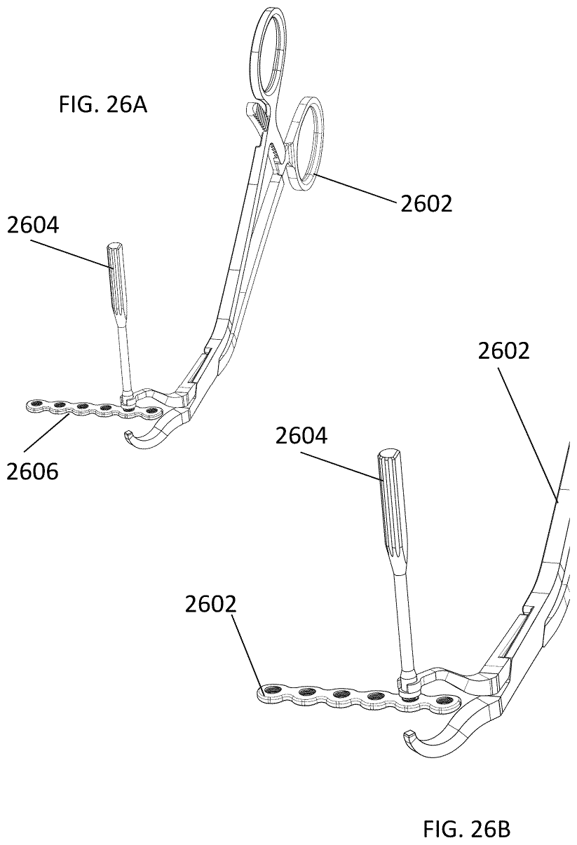

Turning now to FIGS. 26A-26B, similar to the concepts provided above, the a recon implant holder 2602 utilize a central shaft 2604 which connects implant holder 2602 to an implant 2606 and allows for application of a compressive load. This embodiment may use any of the locking holes of plate 2602 rather than a specific implant holder connection point. This embodiment allows a user to select any locking hole on the subject implant to be used for connection to the implant holder, and place the plate screw at any other location on the implant.

Advantages of the above concepts include better fixation of rib fractures and osteotomies, with preference to treatment of osteotomies. The rib head anchors enable fixation in ribs which have been resected close to the transverse process, which would be challenging to fixate using conventional trauma plating methods. Similarly, the low profile of the splint plates allow fixation near a rib head.

The concepts and described here offer significant advantages in treating deformities of the proximal ribs, such as rib hump, but also may be useful in the correction of general chest wall and rib deformities. The symmetric longitudinal member allows contouring in any or multiple planes to achieve a desired outcome. Such contouring is challenging using traditional trauma-plating type devices. The longitudinal member also allows for compression and distraction along the length of the rib to facilitate fracture reduction or direct chest wall expansion. Similarly, the modular nature of the devices described here allows the use of any of multiple techniques, to accommodate the specific surgical needs of the doctor and the anatomy of the patient.

These and other advantages of the present disclosure will be apparent to those skilled in the art from the foregoing specification. Accordingly, it will be recognized by those skilled in the art that changes or modifications may be made to the above-described embodiments without departing from the broad inventive concepts. It should therefore be understood that this disclosure is not limited to the particular embodiments described herein, but is intended to include all changes and modifications that are within the scope and spirit of the disclosure as defined in the claims.

What is claimed is:

1. A system for treating a fracture of a rib, said system comprising:
a plate adapted to be attached to the rib, the plate including a plurality of bone fastener holes, each of the plurality of bone fastener holes configured to receive a bone fastener;
an implant holder having an inferior surface configured to engage an inferior surface of the rib and superior surface;
a drill guide including one or more cannulas and a first threaded portion; and
an inserter instrument having a central shaft and a distal tip, wherein the distal tip is configured to engage bone, and wherein the inserter instrument has a second threaded portion configured to engage and mate with the first threaded portion.

2. The system of claim 1, wherein the inserter instrument has a third threaded portion configured to engage and mate with a fourth threaded portion on the plate.

3. The system of claim 1, wherein the central shaft of the inserter instrument is configured to is configured to engage with the superior surface of the implant holder.

4. The system of the claim 1, wherein the implant holder is a forcep-style instrument.

5. The system of claim 4, wherein the inferior portion of the implant holder is a bottom forcep jaw.

6. The system of claim 5, wherein the superior portion of the implant holder has features that engage the central shaft and are sized to allow disengagement along certain portions of the central shaft.

7. The system of claim 1, wherein the distal tip of the inserter instrument is a sharp point.

8. The system of claim 1, wherein the one or more cannulas are configured to align with one or more of the plurality of bone fastener holes.

9. The system of claim 1, wherein the implant holder is configured to apply a compressive load to the implant via the central shaft.

10. The system of claim 1, wherein the drill guide has pins configured to mate with one or more features on the plate.

11. A system for treating a fracture of a rib, said system comprising:
a plate including a plurality of bone fastener holes;
at least one bone fastener configured to be received in one of the plurality of bone fastener holes;
an implant holder having an inferior surface configured to engage an inferior surface of the rib and a superior surface;
a drill guide including one or more cannulas and a first threaded portion; and
an inserter instrument having a central shaft and a distal tip, wherein the distal tip is configured to engage bone, and wherein the inserter instrument has a second threaded portion configured to engage and mate with the first threaded portion.

12. The system of claim 11, wherein the inserter instrument has a third threaded portion configured to engage and mate with a fourth threaded portion on the plate.

13. The system of claim 11, wherein the central shaft of the inserter instrument is configured to is configured to engage with the superior surface of the implant holder.

14. The system of the claim 11, wherein the implant holder is a forcep-style instrument.

15. The system of claim 14, wherein the inferior portion of the implant holder is a bottom forcep jaw.

16. The system of claim 15, wherein the superior portion of the implant holder has features that engage the central shaft and are sized to allow disengagement along certain portions of the central shaft.

17. The system of claim 11, wherein the distal tip of the inserter instrument is a sharp point.

18. The system of claim 11, wherein the one or more cannulas are configured to align with one or more of the plurality of bone fastener holes.

19. The system of claim 11, wherein the implant holder is configured to apply a compressive load to the implant via the central shaft.

20. The system of claim 11, wherein the drill guide has pins configured to mate with one or more features on the plate.
